(19) United States
(12) Patent Application Publication
(10) Pub. No.: US 2015/0375005 A1
(43) Pub. Date: Dec. 31, 2015

(54) THERAPEUTIC ELECTROMAGNETIC FIELD

(71) Applicant: BrainQ Technologies Ltd, Jerusalem (IL)

(72) Inventor: Yaron SEGAL, Jerusalem (IL)

(73) Assignee: BrainQ Technologies Ltd.

(21) Appl. No.: 14/318,715

(22) Filed: Jun. 30, 2014

(51) Int. Cl. A61N 2/02; A61N 2/00

(52) U.S. Cl. CPC A61N 2/02; A61N 2/006

(57) ABSTRACT

A device for generating a therapeutic electromagnetic field includes at least one conducting coil for placement in the vicinity of a patient. A signal generator is controllable to cause an electrical current to flow through the coil to produce a substantially spatially homogeneous electromagnetic field in at least a part of the patient. The electromagnetic field varies with a frequency that is selectable to substantially match a characteristic operational frequency of a nervous system of the patient.

### FIELD OF THE INVENTION

The present invention relates to generation of a therapeutic electromagnetic field.

### BACKGROUND OF THE INVENTION

Neuroplasticity, or brain plasticity, is the ability of the brain and nervous system to change in response to experience and use. Neuroplasticity enables the brain to learn and remember, as well as to recover function after injury.

Application of time-varying electromagnetic fields has been demonstrated as facilitating restoration of function in the brain and nervous system after trauma, stroke, and spinal cord injury.

Brain activity, e.g., as measured by electroencephalography (EEG) may be classified into several frequency ranges. Delta waves, in the approximate range of 0 to 4 Hz, are associated with subconscious activity and are apparent during deep, dreamless, sleep. Theta waves, waves, in the approximate range of 4 Hz to 8 Hz, are associated with activities related to idleness (e.g., meditation), drowsiness, and deep-brain activity such as memory. Alpha waves, in the approximate range of 8 Hz to 13 Hz, appear apparently during relaxed activity of the central nervous system. The alpha wave range also includes the mu waves (approximately 9 Hz to 11 Hz) that are generally associated with mirror cells that enable imitative behavior and empathy to others. Beta waves, in the approximate range of 13 Hz to 30 Hz, appear during alert brain activity such as active thinking, movement, and similar activities. Gamma waves, in the approximate range of 25 Hz to 100 Hz, generally clearly detected up to 40 Hz, are generally associated with cognitive activity.

### SUMMARY OF THE INVENTION

There is thus provided, in accordance with an embodiment of the present invention, a device for generating a therapeutic electromagnetic field, the device including: at least one conducting coil; and a signal generator that is controllable to cause an electrical current to flow through the at least one coil to produce a substantially spatially homogeneous electromagnetic field in at least a part of the patient, the electromagnetic field varying with a frequency that is selectable substantially match a characteristic operational frequency of a nervous system of the patient.

Furthermore, in accordance with an embodiment of the present invention, wherein the at least one conducting coil includes at least two coils.

Furthermore, in accordance with an embodiment of the present invention, the at least two coils are coaxial.

Furthermore, in accordance with an embodiment of the present invention, the at least two coils are longitudinally arranged so as to produce the electromagnetic field in a region of space that is sufficiently large to include the at least a part of the patient.

Furthermore, in accordance with an embodiment of the present invention, the electromagnetic field is substantially spatially homogenous in the region of space.

Furthermore, in accordance with an embodiment of the present invention, a central space of each coil of the at least two coils is sufficiently large to accommodate the at least a part of the patient.

Furthermore, in accordance with an embodiment of the present invention, the central space of each coil is sufficiently large to accommodate a support of the at least a part of the patient.

Furthermore, in accordance with an embodiment of the present invention, the support includes a bed or a chair, and the at least a part of the patient includes the entire body of the patient.

Furthermore, in accordance with an embodiment of the present invention, the central space is sufficiently large to accommodate the patient when sitting.

Furthermore, in accordance with an embodiment of the present invention, the central space is sufficiently large to accommodate a volume within which the patient is enabled to move freely.

Furthermore, in accordance with an embodiment of the present invention, the at least two coils form a Helmholtz coil arrangement.

Furthermore, in accordance with an embodiment of the present invention, the at least one coil is wearable by the patient.

Furthermore, in accordance with an embodiment of the present invention, the signal generator is portable.

Furthermore, in accordance with an embodiment of the present invention, a strength of the electromagnetic field is less than 10 gauss.

Furthermore, in accordance with an embodiment of the present invention, the device further includes a controller to control operation of the signal generator.

Furthermore, in accordance with an embodiment of the present invention, the controller is further configured to select a coil from the at least one coil through which the current is caused to flow.

Furthermore, in accordance with an embodiment of the present invention, the characteristic operational frequency is between 0.01 Hz and 40 Hz.

There is further provided, in accordance with an embodiment of the present invention, a method for treating a nervous system of a patient, the method including operating a signal generator to cause an electric current to flow through at least one coil to produce a substantially spatially homogeneous electromagnetic field in at least a part of the patient, the electromagnetic field varying with a frequency that is substantially equal to a characteristic operational frequency of the nervous system.

Furthermore, in accordance with an embodiment of the present invention, the characteristic operational frequency is between 0.01 Hz and 40 Hz.

Furthermore, in accordance with an embodiment of the present invention, the method includes placing the at least a part of the patient within a central space of the at least one coil, or placing the at least one coil adjacent to the part of the patient.

Furthermore, in accordance with an embodiment of the present invention, the method includes generating the electromagnetic field in a volume sufficiently large to include an entire body of the patient

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1A is a schematic illustration of a system for therapeutic electromagnetic field generation, in accordance with an embodiment of the present invention.

FIG. 1B is a schematic illustration of a coil of the system shown in FIG. 1A.

FIG. 2 is a schematic illustration of coils of a system for applying a therapeutic electromagnetic field to a single body region, in accordance with an embodiment of the present invention.

FIG. 3 is a schematic illustration of coils of a system for applying a therapeutic electromagnetic field to a sitting patient, in accordance with an embodiment of the present invention.

FIG. 4 is a schematic illustration of coils of a system for applying a therapeutic electromagnetic field to a region of space, in accordance with an embodiment of the present invention.

FIG. 5 is a schematic illustration of a portable system for therapeutic electromagnetic field generation, in accordance with an embodiment of the present invention.

FIG. 6 is a schematic illustration of coils of a system for applying an external therapeutic electromagnetic field, in accordance with an embodiment of the present invention.

FIG. 7 is a flowchart depicting a method of treatment using a therapeutic electromagnetic field, in accordance with an embodiment of the present invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, us of the conjunction “or” as used herein is to be understood as inclusive (any or all of the stated options).

Embodiments of the invention may include an article such as a computer or processor readable medium, or a computer or processor non-transitory storage medium, such as for example a memory, a disk drive, or a USB flash memory, encoding, including or storing instructions, e.g., computer-executable instructions, which when executed by a processor or controller, carry out methods disclosed herein.

In accordance with an embodiment of the present invention, a system, apparatus, or device is configured to produce a time-varying electromagnetic field that varies with a frequency that is suitable for producing a therapeutic effect in a patient's nervous system. As used herein, the terms, “treat,” “treatment,” and “therapeutic” are used to indicate any effect on the nervous system of the patient's nervous system and body, and not necessarily treatment of a disease, injury or other disorder. For example, the terms, “treat,” “treatment,” and “therapeutic” may refer to treatment of a medical condition (e.g., to be understood as including any mild or sever psychological or sociological condition, or to reducing pain), to other health-related benefits (e.g., to maintain, enhance, or prevent deterioration of function of the patient's body or psychological state), for esthetic purposes (e.g., stimulating muscles to achieve a desired body shape or skin tone), for mental effects (e.g., improvement or maintenance of cognitive abilities, to improve skills related to social interactions, to effect a mood change, for inducing a pleasurable feeling or state of mind), for training purposes (e.g., sport-related or other muscle training, mental training, improvement of a skill, or other training or learning), or other effects. Medical treatment may include restoring nerve function, e.g., repairing damaged connections among nerves. Any person to whom the electromagnetic field is applied, whether for medical or for other purposes, is herein referred to as a patient.

As used herein, an electromagnetic field is used to refer to one or both of a time varying magnetic field that is created by application of an electric field (e.g., to a coil, antenna, or other conductor), or to a time varying a magnetic field that induces an electric field or current in a material (e.g., such as of the nervous system). Electromagnetic field strength is herein defined in units of magnetic field strength (e.g., gauss).

A signal generator may be configured to provide a time-varying signal with a selected frequency. The produced signal is applied to one or to a plurality of electrically conducting coils or solenoids to cause a corresponding time-varying current to flow through each of the coils. Coils through which current is made to flow are herein referred to as being activated. As a result of activation, the coils may generate an electromagnetic field. For example, a spatially uniform time-varying electromagnetic field may be generated in a region between two longitudinally adjacent coils. A frequency of the time-varying electromagnetic field is that of the generated signal. The selected frequency may correspond to a frequency of a time varying electromagnetic field that is a characteristic operational frequency of a function or region of the nervous system, that is otherwise known or suspected to have a therapeutic effect on the nervous system, or that is being tested for a therapeutic effect on the nervous system. For example, the frequency may be in the range of 0.01 Hz to 100 Hz (e.g., corresponding to frequencies of brain activity). In some cases, the frequency may be in the range of 0.01 Hz to 40 Hz or 3 Hz to 40 Hz.

For example, a characteristic operational frequency of the nervous system may correspond to an operational frequency that is characteristic of an activity of the nervous system. Such an activity may include, for example, a frequency of electrical impulses in the brain or through neural networks, of chemical pulses across synapses, or other activity.

The coils for producing the electromagnetic field may be arranged in such a manner that is suited for application to a particular patient. The size and longitudinal spacing of the coils may be selected so as to produce a time-varying electromagnetic field of sufficient magnitude, spatial uniformity, or other properties so as to have the desired therapeutic effect. A size of the region in which the time-varying electromagnetic field is to be generated may also be determined in accordance with, for example, a size of the region to be treated, a size of support structure (e.g., constructed of non-electromagnetic material) for supporting the patient during treatment, a position of the patient during treatment (e.g., lying, sitting, standing, or another position), a degree of cooperation that is expected from the patient during treatment (e.g., ability to remain in one place during treatment, fears of enclosed places, or other traits that may affect cooperation), interference from other equipment in the vicinity, or other considerations.

The coils may be longitudinally arranged along a common axis to generate an electromagnetic field in a region of space that includes a section of the body that is to be treated. Thus, when treating the brain or head, a Helmholtz coil arrangement of two or more substantially identical coaxial coils may be arranged to bound the head of the patient (e.g., one at the top of the head and one at the neck). The coils may have diameters that are suitable for placement about the patient's head and any platform or support structure that is used to support the head or to hold it in place during treatment. Coils of different diameters may be provided for patients with differently sized heads. When the spinal cord or other part of the patient's trunk or body (e.g., thoracic, abdominal, or pelvic region) is to be treated, coils may be arranged coaxially along the length of the section of the patient's trunk that is to be treated. The diameters of the coils may be sufficient to accommodate a bed or other support on which the patient is lying during treatment. Similarly, arrangements of coils may be designed for treatment of a single limb (arm or leg) of the patient, and for a sling or splint that supports the limb.

A patient may be treated while in a sitting position. (Sitting as used herein, includes sitting upright, and to a partially reclining position between sitting upright and lying or fully reclining on a flat horizontal surface.) In some cases, a period of time for application of the electromagnetic field treatment may be longer than a time during which the patient could comfortably lie still. For example, injuries, back disorders, or other circumstances disorders may present a difficulty in having a patient lie within a confined space for an extended period of time. In some cases, e.g., when treatment is to be applied during the course of a long period of time (e.g., either continuously or requiring a long series of periodic treatments), enabling a patient to be treated a while being seated could increase the patient's comfort. For example, a seated patient could read, write, watch a screen, play a game, or engage in other activities that could be difficult for a patient in a lying position (either prone, supine, or on the side). An appropriately sized cylindrical chamber may be configured to accommodate the shape of the patient while seated. Walls of the cylindrical chambers may include coaxial coils whose diameter and spacing and may be such as to produce a substantially spatially homogeneous (e.g., with local variations in uniformity being less than a maximum allowed deviation) time varying electromagnetic field within the space of the chamber. The coils may be exposed or may be concealed within the walls of the chamber. The spatial homogeneity may allow the patient at least limited freedom of movement within the chamber without adversely affecting the efficacy of the treatment.

In some cases, a patient may be treated while being allowed to move about freely within a room-sized chamber. For example, various circumstances may preclude patient from being enclosed within a chamber. For example, it may be difficult or traumatic to a child or to a person with claustrophobia or an autistic disorder to be enclosed within chamber or space of limited size. In some cases, e.g., in the case of a patient who is a child, it may be desirable that two or more people be present within the chamber. A presence of another person may assist in allaying fears may provide a diversion for the patient. A floor, e.g., of a non-electromagnetic material, may be placed within the chamber to enable the patients or others within the chamber to move about freely within the chamber. The coils are of such size and spacing as to produce a spatially homogeneous time-varying electromagnetic field within the entire space of the chamber.

In some cases, a patient may be treated by a portable therapeutic electromagnetic field system. For example, the coils may be incorporated into a collar, sleeve, vest, band, belt, sash, hat, or otherwise into a wearable or portable article that may be worn by the patient. In some cases, the coils may be configured to generate a therapeutic electromagnetic field in a region external to (e.g., adjacent to) the coils.

A frequency of a generated therapeutic electromagnetic field may be selected for a particular treatment. For example, a frequency may be appropriate to a general region or functionality of the brain or of the nervous system (e.g., motor, memory, or other general functionality). A frequency may be appropriate to a particular region or functionality of the brain or nervous system (e.g., movement of a particular limb or muscle, particular type of cognitive ability, or other particular functionality). In some cases, a plurality of signals of different frequencies may be concurrently applied to different coils of the therapeutic electromagnetic field system. In this manner, more than one region or functionality may be treated concurrently. In some cases, a plurality of signals of different frequencies may be concurrently applied to a single set of coils of the therapeutic electromagnetic field system. The time variation of the therapeutic electromagnetic field may thus be a superposition of the various applied frequencies.

Field strength of the electromagnetic field (e.g., the amplitude of the electromagnetic field) may be below 100 gauss. In some cases, the field strength may be below 50 gauss or below 10 gauss. In some cases, the field strength may be below 5 gauss (e.g., to conform to standards that are intended to eliminate possible interference with pacemakers). Field strength may be below 0.5 gauss, except where the intention is to induce a palpable or sensible effect on the nervous system.

A therapeutic electromagnetic field system may be utilized to selectively exercise muscles of the patient. For example, an electromagnetic field of a particular frequency may be applied to cause the nervous system to tighten a particular muscle (e.g., by an amount that does not result in noticeable movement). The tightening of the muscles may be beneficial for rehabilitative purposes, for weight loss, or for other health or esthetic purposes. Concurrent application of fields of different frequencies (e.g., by different coils or by the same coil) may enable simultaneous activation of different muscles.

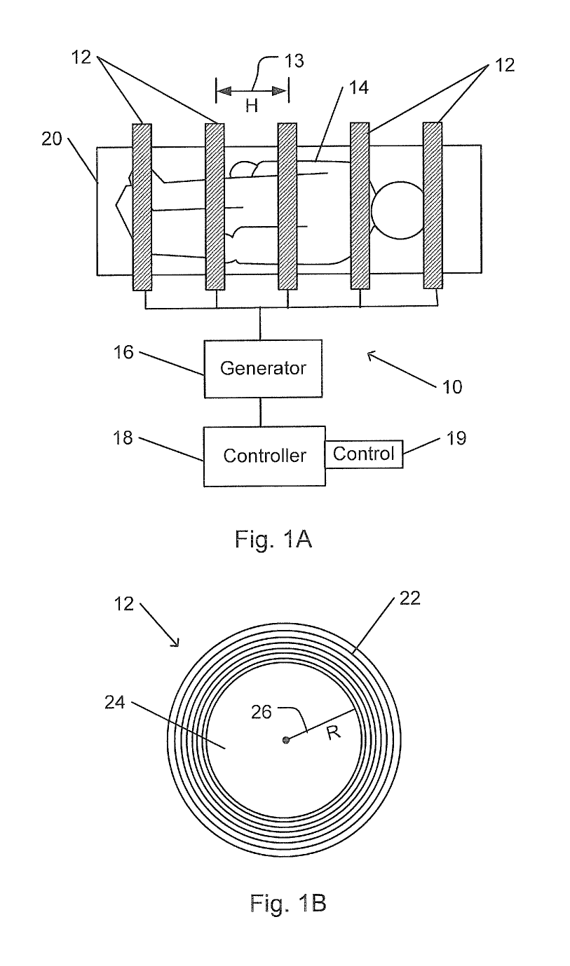

FIG. 1A is a schematic illustration of a system for therapeutic electromagnetic field generation, in accordance with an embodiment of the present invention. FIG. 1B is a schematic illustration of a coil of the system shown in FIG. 1A.

Therapeutic electromagnetic field system 10 may be configured to provide a therapeutic electromagnetic field that may be applied to a patient 14. The therapeutic electromagnetic field may be applied to the entire body of patient 14, a single region of patient 14 (e.g., head, thorax, abdomen), or a limb of patient 14 (e.g., arm, hand, finger, leg, foot, or other limb). Patient 14 may represent a human patient, or may represent an animal that is to be treated. The part of patient 14 that is to be treated (e.g., entire body, region, or limb), is herein referred to as a target region of patient 14. Therapeutic electromagnetic field system 10 may be preconfigured, e.g., by a manufacturer or installer to treat a particular target region of patient 14. Therapeutic electromagnetic field system 10 may be configured may be configurable by an operator to treat different target regions of patient 14. For example, one or more components of therapeutic electromagnetic field system 10 may be replaceable, moveable, adjustable, or otherwise modifiable in order to adapt therapeutic electromagnetic field system 10 to treatment of a particular target region of patient 14.

An operator may include a healthcare professional, a technician, another professional (e.g., trainer, instructor, coach, beautician, or other type of professional), an untrained person or the patient. For example, therapeutic electromagnetic field system 10 may be installed in a hospital, clinic, office, rehabilitation or treatment facility, or other location or setting that is staffed by healthcare professionals. Therapeutic electromagnetic field system 10 may be installed in a gym or other sports facility, spa, salon, or other facility that is staffed by technicians, or other non-healthcare professionals. Therapeutic electromagnetic field system 10 may include a self-treatment device, e.g., for use by the patient in the patient's home. Operation of a therapeutic electromagnetic field system 10 for self-treatment may be limited to operation in accordance to a single treatment protocol (e.g., as prescribed by a healthcare professional), or may be limited to limited number or approved (e.g., by a healthcare professional, or by a manufacturer or distributor) treatment protocols.

Therapeutic electromagnetic field system 10 includes two or more coaxial coils 12. Each coaxial coil 12 is in the form of an annular electrical conductor. For example, the circular conductor may include winding 22 of a conducting wire about a circular central space 24. Coaxial coil 12 may be characterized by a characterizing radius 26 of length R. For example, characterizing radius 26 may represent a radius of circular central space 24, an outer radius of coaxial coil 12, or a representative (e.g., average) radius of winding 22. Adjacent coaxial coils 12 may be separated by a separation distance 13 of length H. For example, separation distance 13 may represent a center-to-center distance between adjacent coaxial coils 12.

Length R of characterizing radius 26 is sufficient to enable placement of patient 14 (e.g., a target region of patient 14) and support platform 20 within central space 24. Support platform 20 may be configured to support all or part of patient 14. For example, support platform 20 may be configured to support a target region of patient 14. Support platform 20 may be moveable, foldable, rotatable, or otherwise adjustable to support a particular target region of patient 14. Support platform 20 may be adjustable (e.g., translatable) to enable sequential treatment of different target regions of patient 14. For example, different target regions may be sequentially positioned at a particular point relative to coaxial coils 12.

For example, when the target region includes the entire body of patient 14, support platform 20 may be configured to support the entire body of patient 14. In this case, support platform 20 may represent a bed, chair, cot, cradle, stretcher, gurney, or other structure that may be used to support the entire body of patient 14. When that target region is a particular region or limb of patient 14, support platform 20 may be configured to support that region or limb. For example, support platform 20 may represent a head support, sling, splint, or other structure that may be used to support a region or limb of patient 14.

Coaxial coils 12 are configured to produce an electromagnetic field within and outside of central space 24 when a current flows through winding 22. The general direction of the produced electromagnetic field within central space 24 is approximately perpendicular to a plane (e.g., a midplane) of central space 24.

Signal generator 16 is configured to generate an electrical current that may flow through winding 22 of each coaxial coil 12. Signal generator 16 may be configured to produce a sinusoidal electrical current or a signal that is describable by another functional form. Signal generator 16 may be operated to produce a monotonic signal of a predetermined frequency or a signal that represents a series or weighted sum of monotonic signals of different frequencies. Signal generator 16 may be configured to generate electrical signals within a frequency range that is known of a therapeutic electromagnetic field, e.g., of a range of characteristic operational frequencies of the nervous system.

Operation of signal generator 16 may be controlled by controller 18. For example, controller 18 may include one or more operator-operable controls 19 that may be operated by an operator to control operation of signal generator 16. For example, a user or operator may operate operator-operable controls 19 of controller 18 to select treatment parameters a predefined treatment protocol. Treatment parameters may include, for example, a frequency, amplitude, or both of a generated signal, a duration of the signal (or a start or stop time for the signal), which coaxial coils 12 to operate (e.g., connect to signal generator 16), or other treatment parameters or properties of the generated signal. An operator may operate operator-operable controls 19 to indicate an identity of a patient to be treated. Controller 18 may include a processor, such as of computer, in communication with a memory unit and configured to operate in accordance with programmed instructions. The processor may be configured to automatically operate signal generator 16 at predetermined times, or in response to an event, such as operation of an operator-operable control. Controller 18 may be configured to communicate with a remote device. For example, an operator of controller 18 may enter identifying data regarding a patient. Controller 18 may then communicate with a remote server or other device to retrieve predetermined treatment parameters or a treatment protocol for that patient.

Electrical power for operation of signal generator 16, controller 18, or both, may be provided from power mains, or may be provided by a battery or other portable power source (e.g., generator, solar cell array, or other portable power source).

Length R of characterizing radius 26 and distance H between adjacent coaxial coils 12 may be selected so as to generate a spatially homogeneous electromagnetic field in which patient 14 is placed. For example, coaxial coils 12 may be arranged in a Helmholtz coil arrangement (e.g., with H R).

Signal generator 16 may be operated to cause a current to flow through winding 22 of all of coaxial coils 12, or of a subset of two or more of coaxial coils 12. For example, generation of the therapeutic electromagnetic field may be limited to a single part of the body of patient 14. Thus, signal generator 16 may be configured (e.g., by selectively operating one or more switches) such that current only flows through, or activates, those coaxial coils 12 that surround the body part (e.g., head, spine, or another part) to be treated.

In some cases, those coaxial coils that surround an injured or impaired body part plus adjacent body parts (e.g., with frequencies appropriate to those body parts). Treatment of adjacent body parts may be beneficial to continuity of the neural connections between that adjacent body parts. For example, in the case of head injury, coaxial coils 12 that surround the head and the cervical and thoracic spine may be activated. In the case of back injury, all of coaxial coils 12 may be activated to create or repair neural continuity throughout the body. When the location of a spine injury is known, treatment may be limited to those limbs whose neural connections to the spinal cord are below the point of the injury. Treatment may be applied sequentially at the various frequencies that are appropriate to treating the various body parts.

An amplitude or strength of the generated electromagnetic field may be determined by one or more factors as lengths R and H, number of coaxial coils 12, number of windings 22, a current flowing through winding 22, nearby materials, or other factors.

In accordance with an embodiment of the present invention, a therapeutic electromagnetic field may be applied to a single region or limb of a patient's body.

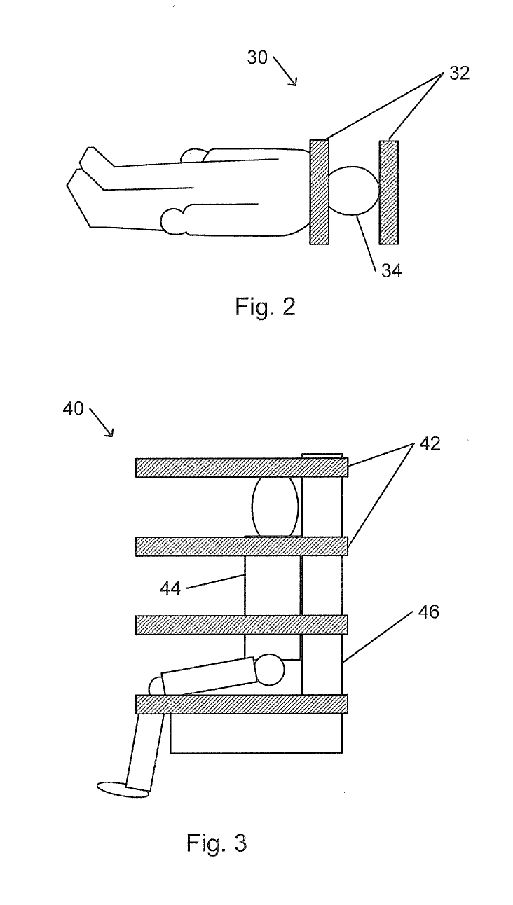

FIG. 2 is a schematic illustration of coils of a system for applying a therapeutic electromagnetic field to a single body region, in accordance with an embodiment of the present invention.

Body region treatment system 30 may be configured to apply a therapeutic electromagnetic field to a head 34 as shown, or to another region or limb of the patient's body. For example, head coils 32 may be of such size and placement to generate a desired electromagnetic field at head 34.

For example, body region treatment system 30 may be configured to apply treatment to a head 34 to provide a treatment for a condition or disorder such as autism, cerebral palsy, familial dysautonomia, or conditions that involve neural degeneration in the brain (e.g., Parkinson's disease, Alzheimer's disease, multiple sclerosis, or other conditions involving the brain or nervous system), as well as head trauma, stroke, or other head injuries.

For example, a treatment may be applied in an emergency situation without a detailed evaluation. For example, if injury is detected to a particular region or function of the brain, a treatment may be applied that is general to the identified region or function. For example, in the case of detected injury to an area of the brain related to motor movements, head coils 32 may be operated to generate an electromagnetic field with a characteristic operational frequency of the motor-related regions (e.g., 15 hertz) or of the central nervous system (e.g., 7.82 hertz). If the injury is to an area of the brain related to memory, head coils 32 may be operated to generate an electromagnetic field with a characteristic operational frequency of the memory-related regions (e.g., 3.75 hertz).

Treatment may be applied after emergency treatment, e.g., by a hospital or rehabilitation facility after a detailed evaluation of the patient. The treatment in this case may be specific to the results of the injury. For example, if an injury is detected to an area of the brain related to movement of the left leg, an electromagnetic field may be generated with a characteristic operational frequency of movement of the left leg (e.g., 28 hertz). If the injury is to an area of the brain related to movement of the right leg, the electromagnetic field frequency may a characteristic operational frequency of movement of the right leg (e.g., 22 hertz).

As treatment continues (e.g., follow treatment in an outpatient facility), the frequency of the generated electromagnetic field may be selected to be specific to whatever particular functionality (e.g., a particular movement of a limb) that remains impaired.

In accordance with an embodiment of the present invention, a therapeutic electromagnetic field system may be configured to be applied to patients in various types of body positions (lying, standing, sitting, crouching, reclining, or other body positions), or that are being supported by variously configured support platforms (beds, cots, chairs, slings, gurneys, or other support platforms).

FIG. 3 is a schematic illustration of coils of a system for applying a therapeutic electromagnetic field to a sitting patient, in accordance with an embodiment of the present invention.

Seated patient therapeutic electromagnetic field system 40 is configured to apply a therapeutic electromagnetic field to a seated patient 44. For example, chair 46 may be constructed of nonmagnetic materials. Chair 46 may be configured to support seated patient 44 in a sitting or similar position. Coils 42 are sufficiently large to entirely surround a part of seated patient 44 that is to be treated. For example, coils 42 may have a central space that is sufficiently large to surround the torso, thighs, and knees of sitting patient 46.

Using seated patient therapeutic electromagnetic field system 40 to treat a sitting patient 46 may be advantageous over treatment of a fully reclining or lying patient. In some cases, injuries may prevent a patient from lying, or may cause lying to be uncomfortable or painful. While seated, a sitting patient 46 may be able to engage in wider variety of activities that could a lying patient. The activities may have a therapeutic purpose. For example, engaging in the activities may promote a therapeutic effect. Engaging in the activities may contribute to evaluation of a therapeutic effect of application of the electromagnetic field to seated patient 44 (cognitive functional testing). Activities by a seated patient 44 may make the experience of the treatment more pleasant. For example, engagement in activities may reduce feelings of tedium or boredom, or may by enable seated patient 44 to engage in activities related to employment, hobbies, housekeeping duties, or other activities.

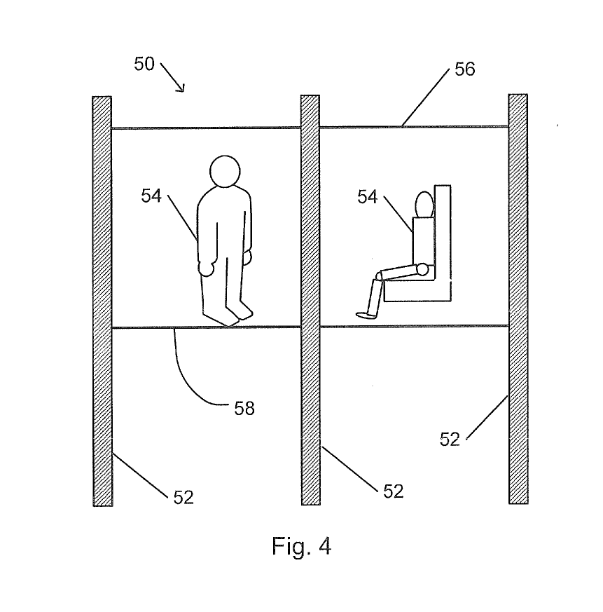

FIG. 4 is a schematic illustration of coils of a system for applying a therapeutic electromagnetic field to a volume of space, in accordance with an embodiment of the present invention.

Volume electromagnetic field system 50 is configured to apply a therapeutic electromagnetic field to one or more patients that are within volume 56. Volume 56 may be configured to accommodate two or more unrestrained patients 54. Coils 52 are configured to generate an electromagnetic field throughout volume 56. Thus, when being treated, unrestrained patients 54 may move freely within the confines of volume 56, and may shift their positions (e.g., stand or sit).

For example, volume 56 may represent an enclosed chamber with a floor 58. Coils 52 may be visible from within volume 56, or may be concealed from view. For example, coils 52 may be located outside the enclosure (e.g., floor 58, and walls and ceiling of the enclosure). Coils 52 may be disguised as other structure with a decorative or functional purpose, or may be concealed by such structure.

Floor 58 may be placed to provide a suitable support surface for unrestrained patients 54. For example, an area of floor 58 that is placed near a diameter of coils 52 may be larger than an area of a floor 58 that is placed elsewhere within coils 52.

Using volume electromagnetic field system 50 to treat unrestrained patients 54 may be advantageous, e.g., especially in treating brain disorders. For example, a patient that is to be treated for an extended period of time may have difficulty for sitting or lying throughout the period of treatment. Some patients, e.g., children or others with conditions that cause difficulties with sitting still or lying still, may have difficulty staying in one place for even short periods of time. Some patients (e.g., young children, claustrophobic individuals, or others) may require the close presence of another person (e.g., parent, relative, or acquaintance) to overcome any fear of the therapeutic electromagnetic field generation device.

In accordance with an embodiment of the present invention, a therapeutic electromagnetic field system may be configured to be portable. A portable therapeutic electromagnetic field system may be designed to be worn by a patient. For example, a portable therapeutic electromagnetic field system may be configured to a apply a therapeutic electromagnetic field to a single body part of a patient

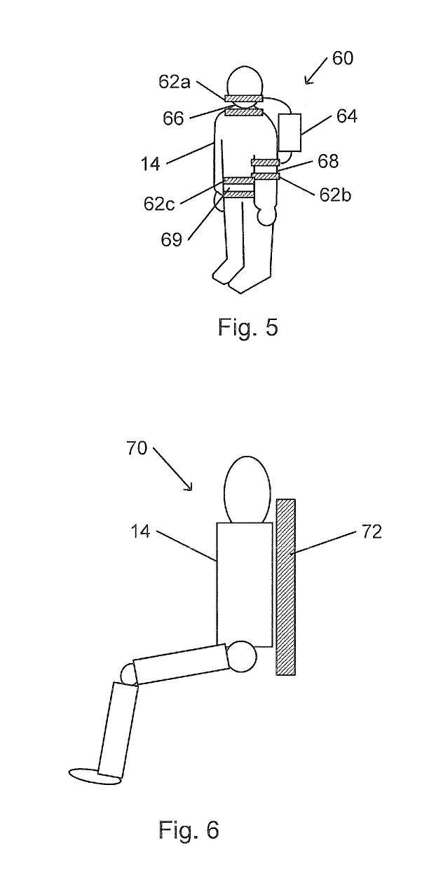

FIG. 5 is a schematic illustration of a portable system for therapeutic electromagnetic field generation, in accordance with an embodiment of the present invention.

Portable therapeutic electromagnetic field system 60 may be worn, or otherwise carried, by a patient 14. For example, portable therapeutic electromagnetic field 60 system may include one or more of neck coils 62a to be worn as a collar on neck 66 of patient 14, limb coils 62b to worn (e.g., as a cuff or otherwise) on limb 68 of patient 14, and trunk coils 62c to be worn (e.g., as a sash, brace, or otherwise) on a trunk 69 of patient 14. Operation of neck coils 62a, of limb coils 62b, or of trunk coils 62c may apply a therapeutic electromagnetic field to neck 66, limb 68, or trunk 69 of patient 14.

Neck coils 62a, of limb coils 62b, or of trunk coils 62c may be operated by portable operation unit 64. For example, portable operation unit 64 may be worn as a backpack, carried, wheeled, incorporated into the coils (e.g., on an outer perimeter of neck coils 62a, of limb coils 62b, or of trunk coils 62c), or otherwise worn or transported, by patient 14. Portable operation unit 64 may include one or more of a signal generator, a controller, an operator-operable control, a portable power supply, and any other component. Portable operation unit 64 may include one or more compartments for carrying components or spare parts of portable therapeutic electromagnetic field system 60.

For example, neck coils 62a may be operated to stimulate the muscles related to swallowing or speech. Limb coils 62b or trunk coils 62c may be operated to stimulate muscles related to limb 68 or trunk 69.

In some cases, a therapeutic electromagnetic field system may include a single coil or set of coils that are configured to generate an external therapeutic electromagnetic field. The external therapeutic electromagnetic field that is applied is generated externally to all coils of the set, and not between coils.

FIG. 6 is a schematic illustration of coils of a system for applying an external therapeutic electromagnetic field, in accordance with an embodiment of the present invention.

Externally generated therapeutic electromagnetic field system 70 includes external coil 72. External coil 72 may represent a single coil, or may represent a set of component coils. Component coils of external coil 72 may be arranged to produce an electromagnetic field with a desired spatial variation. External coil 72 may be flexible to enable external coil 72 to be worn by a patient 14, or otherwise conform closely to the shape of patient 14. Component coils of external coil 72 need not be arranged coaxially.

External coil 72 is configured to generate a therapeutic electromagnetic field that may be applied to a patient 14. For example, the therapeutic electromagnetic field may be applied to a back of patient 14, or to another body part of patient 14.

A method for treatment with a therapeutic electromagnetic field may be performed, e.g., by a controller of a therapeutic electromagnetic field system, e.g., with the assistance of an operator.

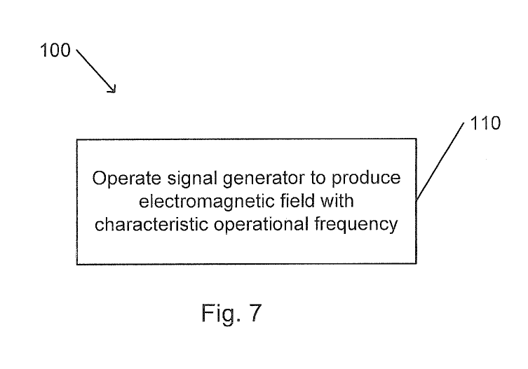

FIG. 7 is a flowchart depicting a method of treatment using a therapeutic electromagnetic field, in accordance with an embodiment of the present invention.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

Treatment method 100 may be performed by a controller of a therapeutic electromagnetic field system. Performance of treatment method 100 may be initiated by an operator of the therapeutic electromagnetic field system. An operator may include a person who is with the patient being treated (e.g., a healthcare professional or practitioner), a remote operator (e.g., via a wired or wireless network connection), or the patient. Performance of treatment method 100 may be initiated automatically by the controller. For example, performance of treatment method 100 may be initiated at predetermined times, or in response to a predetermined event (e.g., a sensor indication of a status or condition of the patient, or another event).

Parameters of a therapeutic electromagnetic field that are appropriate to a treatment may be selected. For example, the parameters may be selected by selection of a particular treatment protocol. The parameters may be selected by an operator of the therapeutic electromagnetic field system. The parameters may be selected in accordance with specific characteristics of the patient (e.g., age, weight, height, sex, preexisting disorders or conditions, diet, habits, body composition, or other characteristics), of a condition to be treated, or of the treatment. The parameters may include: an amplitude or frequency of an electric signal that is to be applied to coils of the therapeutic electromagnetic field system, a selection of coils from a set of coils to which the signal is to be applied, a required strength or degree of spatial homogeneity of the therapeutic electromagnetic, or other characteristics of the applied signal or of the therapeutic electromagnetic field. The parameters may include a time interval during which the therapeutic electromagnetic field is to be generated, or a start or stop time, a repetition rate (e.g., when periods of application of the therapeutic electromagnetic field alternate with periods in which no electromagnetic field is applied), or other parameters. A coil or set of coils may be selected as appropriate to the treatment that is to be applied (e.g., by physically attaching or detaching coils to or from the therapeutic electromagnetic field system, or by operating a switch to select the coils). For example, a frequency may be selected to substantially match a characteristic operational frequency of a nervous system of the patient.

A signal generator may be operated to cause an electric current to flow through the coils to produce a substantially spatially homogeneous electromagnetic field in at the patient or in a part of the patient (block 110). The frequency of the current may be substantially equal to a characteristic operational frequency of the nervous system of the patient. For example, application of the electric signal may be initiated automatically by a controller of the therapeutic electromagnetic field system after parameters are selected. Application of the electric signal may be initiated by a patient or operator of the therapeutic electromagnetic field system. In some cases, application of the electric signal may be contingent on a status of an interlock or other component, or on another condition being met.

1. A device for generating a therapeutic electromagnetic field, the device comprising:
at least one conducting coil; and
a signal generator that is controllable to cause an electrical current to flow through said at least one coil to produce a substantially spatially homogeneous electromagnetic field in at least a part of a patient, the electromagnetic field varying with a frequency that is selectable to substantially match a characteristic operational frequency of a nervous system of the patient.

2. The device of claim 1, wherein said at least one conducting coil comprises at least two coils.

3. The device of claim 2, wherein said at least two coils are coaxial.

4. The device of claim 2; wherein said at least two coils are longitudinally arranged so as to produce the electromagnetic field in a region of space that is sufficiently large to include said at least a part of the patient.

5. The device of claim 2, wherein a central space of each coil of said at least two coils is sufficiently large to accommodate said at least a part of the patient.

6. The device of claim 5, wherein the central space of each coil is sufficiently large to accommodate a support of said at least a part of the patient.

7. The device of claim 6, wherein the support comprises a bed or a chair, and said at least a part of the patient comprises the entire body of the patient.

8. The device of claim 5, wherein the central space is sufficiently large to accommodate the patient when sitting.

9. The device of claim 5, wherein the central space is sufficiently large to accommodate a volume within which the patient is enabled to move freely.

10. The device of claim 2, wherein said at least two coils form a Helmholtz coil arrangement.

11. The device of claim 1, wherein said at least one coil is wearable by the patient.

12. The device of claim 11, wherein the signal generator is portable.

13. The device of claim 1, wherein a strength of the electromagnetic field is less than 10 gauss.

14. The device of claim 1, further comprising a controller to control operation of the signal generator.

15. The device of claim 14, wherein the controller is further configured to select a coil from said at least one coil through which the current is caused to flow.

16. The device of claim 1, wherein the characteristic frequency is between 0.01 Hz and 40 Hz.

17. A method for treating a nervous system of a patient, the method comprising operating a signal generator to cause an electric current to flow through at least one coil to produce a substantially spatially homogeneous electromagnetic field in at least a part of the patient, the electromagnetic field varying with a frequency that is substantially equal to a characteristic operational frequency of the nervous system.

18. The method of claim 17, wherein the characteristic operational frequency is between 001 Hz and 40 Hz.

19. The method of claim 17, comprising placing said at least a part of the patient within a central space of said at least one coil, or placing said at least one coil adjacent to the part of the patient.

20. The method of claim 17, comprising generating the electromagnetic field in a volume sufficiently large to include an entire body of the patient.
